(19) United States
(12) Patent Application Publication
(10) Pub. No.: US 2014/0148446 A1
(43) Pub. Date: May 29, 2014

(54) COMPOUNDS THAT ENABLE ALTERNATIVE MITOCHONDRIAL ELECTRON TRANSFER

(71) Applicants: James W. Simpkins, Fort Worth, TX (US); Yi Wen, Keller, TX (US); Shaohua Yang, Southlake, TX (US)

(72) Inventors: James W. Simpkins, Fort Worth, TX (US); Yi Wen, Keller, TX (US); Shaohua Yang, Southlake, TX (US)

(73) Assignee: University of North Texas Health Science Center, Fort Worth, TX (US)

(21) Appl. No.: 13/825,819

(22) PCT Filed: Sep. 23, 2011
(86) PCT No.: PCT/US11/53101

§ 371 Date: Sep. 1, 2013

Related U.S. Application Data
(60) Provisional application No. 61/385,915, filed on Sep. 23, 2010.

(51) Int. Cl. A61K 31/5415; A61K 45/06

(52) U.S. Cl. CPC A61K 31/5415; A61K 45/06
USPC 514/224.8; 544/37

(57) ABSTRACT

The present invention relates to novel compositions and uses thereof as antioxidants and/or neuroprotective agents for the treatment of medical conditions associated with oxidative stress and/or neural damage, such as, for example, neurological diseases, disorders and trauma, and hence in the treatment of CNS-associated diseases, disorders and trauma.

### CROSS-REFERENCES TO RELATED APPLICATIONS

This Application claims priority from U.S. Provisional Patent Application No. 61/385,915 filed Sep. 23, 2010, which is hereby incorporated by reference as if fully set forth herein.

### STATEMENT REGARDING FEDERALLY-SPONSORED RESEARCH OR DEVELOPMENT

This invention was made with government support under grant numbers R01NS054687, R01NS054651, P01AG22550 and P01AG10485, awarded by the National Institutes of Health; and grants awarded by Alzheimer's Association and Texas Garvey Foundation. The government has certain rights in the invention.

### BACKGROUND OF THE INVENTION

The present invention relates to novel compositions and uses thereof as antioxidants and/or neuroprotective agents for the treatment of medical conditions associated with oxidative stress and/or neural damage, such as, for example, neurological diseases, disorders and trauma, and hence in the treatment of CNS-associated diseases, disorders and trauma.

Oxidative stress may be considered as a disturbance in the equilibrium status of pro-oxidant/anti-oxidant systems in intact cells, and may result from a number of different oxidative challenges, including radiation, metabolism of environmental pollutants and administered drugs, as well as immune system response to disease or infection. When oxidative stress occurs, the pro-oxidant systems outbalance those of the anti-oxidant, which may result in oxidative damage to cell components including lipids, proteins, carbohydrates, and nucleic acids. Mild, chronic oxidative stress may alter the anti-oxidant systems by inducing or repressing proteins that participate in these systems, and by depleting cellular stores of anti-oxidant materials such as glutathione and Vitamin E. Severe oxidative stress may ultimately lead to cell death.

Oxidative stress therefore involves reactive oxygen species (ROS), which have been implicated in the development of many heart and central nervous system (CNS) dysfunctions. Ischemia/reperfusion insults to these organs are among the leading causes of mortality in humans. These insults are caused by complete or partial local occlusions of heart and brain vasculature, by heart stroke or attack, and by cerebral attacks and trauma to the brain. In addition, ROS are involved in artherosclerotic lesions, in the evolution of various neurodegenerative diseases, and are also produced in association to epileptic episodes, in inflammation, in the mechanisms of action of various neurotoxicants, or as side-effects of drugs. Hence, antioxidative agents and drugs constitute a highly sought after target in contemporary drug development and pharmaceutical research.

Chronic degenerative changes, as well as delayed or secondary neuronal damage following direct injury to the CNS, may result from pathologic changes in the brain's endogenous neurochemical systems. Although the precise mechanisms mediating secondary damage are poorly understood, post-traumatic neurochemical changes may include overactivation of neurotransmitter release or re-uptake, changes in presynaptic or postsynaptic receptor binding, or the pathologic release or synthesis of endogenous factors. The identification and characterization of these factors and of the timing of the neurochemical cascade after CNS injury provides a window of opportunity for treatment with pharmacologic agents that scavenge ROS, modify synthesis, release, receptor binding, or physiologic activity of neurotransmitters and other endogenous factors with subsequent attenuation of neuronal damage and improvement in outcome. A number of studies have suggested that modification of post-injury events through pharmacologic intervention can promote functional recovery in both a variety of animal models and clinical CNS injury. Pharmacologic manipulation of endogenous systems by such diverse pharmacologic agents as anticholinergics, excitatory amino acid antagonists, including specifically N-methyl-D-aspartate (NMDA) receptor antagonists, endogenous opioid antagonists, catecholamines, serotonin antagonists, modulators of arachidonic acid, antioxidants and free radical scavengers, steroid and lipid peroxidation inhibitors, platelet activating factor antagonists, anion exchange inhibitors, magnesium, gangliosides, and calcium channel antagonists have all been suggested to potentially improve functional outcome after brain injury.

Neuroprotective strategies, including free radical scavengers, glutamate receptor antagonists, ion channel modulators, and anti-inflammatory agents, have been extensively explored in the last two decades for the treatment of neurological diseases. Unfortunately, none of the neuroprotectants have been proved effective in clinical trails. The present invention demonstrates that methylene blue (MB) functions as an alternative electron carrier, which accepts electrons from NADH and transfers them to cytochrome c, and bypasses complex I/III blockage. While, a de novo synthesized MB derivative, with the redox center disabled by N-acetylation had no effect on mitochondrial complex activities. MB increases cellular oxygen consumption rate and reduces cellular glycolysis in cell cultures. MB is protective against various insults in vitro at low nM concentrations. The data indicates that MB has a unique mechanism and is fundamentally different from traditional antioxidants. MB dramatically attenuates behavioral, neurochemical, and neuropathological impairment in a rat Parkinson's disease model. Rotenone caused severe dopamine depletion in the striatum, which was almost completely rescued by MB. MB rescued the effects of rotenone on mitochondrial complex I-III inhibition and free radical over-production. Rotenone induced a severe loss of nigral dopaminergic neurons, which was dramatically attenuated by MB. In addition, MB significantly reduced cerebral ischemia reperfusion damage in a transient focal cerebral ischemia model. The present invention is directed to the rerouting of mitochondrial electron transfer by MB or similar molecules which provides a novel strategy for neuroprotection against both chronic and acute neurological diseases involving mitochondrial dysfunction.

### SUMMARY OF THE INVENTION

The present invention is directed to compositions comprising methylene blue and similar molecules that can serve as antioxidants and neuroprotectants. The present invention is further directed to the use of compositions comprising methylene blue as antioxidants and/or neuroprotective agents for the treatment of various medical conditions associated with oxidative stress, neurodegeneration and/or neural damage, as well as other medical conditions as is further delineated herein.

According to an additional aspect of the present invention there is provided a pharmaceutical composition which includes, as an active ingredient, the compositions as described herein and a pharmaceutically acceptable carrier.

According to further features in preferred embodiments of the invention described below, the pharmaceutical composition is being packaged in a packaging material and identified in print, in or on the packaging material, for use in the treatment of a medical condition selected from the group consisting of a CNS associated disease, disorder or trauma, an oxidative stress associated disease or disorder, and a disease or disorder in which neuroprotection is beneficial.

According to yet another aspect of the present invention there is provided a use of the compound presented herein for the treatment of a medical condition selected from the group consisting of a CNS associated disease, disorder or trauma, an oxidative stress associated disease or disorder, and a disease or disorder in which neuroprotection is beneficial.

According to further features in preferred embodiments of the invention described below, the oxidative stress associated disease or disorder is selected from the group consisting of atherosclerosis, an ischemia/reperfusion injury, restenosis, hypertension, cancer, an inflammatory disease or disorder, an acute respiratory distress syndrome (ARDS), asthma, inflammatory bowel disease (IBD), a dermal and/or ocular inflammation, arthritis, metabolic disease or disorder and diabetes.

According to still further features in preferred embodiments the CNS associated disease, disorder or trauma is selected from the group consisting of a neurodegenerative disease or disorder, a stroke, a brain injury and/or trauma, multiple sclerosis, amyotrophic lateral sclerosis, Huntington's disease, Parkinson's disease, Alzheimer's disease, Friedrich's ataxia, autoimmune encephalomyelitis, AIDS associated dementia, epilepsy, schizophrenia, pain, anxiety, an impairment of memory, a decreased in cognitive and/or intellectual functions, a deterioration of mobility and gait, an altered sleep pattern, a decreased sensory input, a imbalance in the autonomic nerve system, depression, dementia, confusion, catatonia and delirium.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control.

In addition, the materials, methods, and examples are illustrative only and not intended to be limiting.

As used herein, the singular form “a,” “an,” and ^{the} include plural references unless the context clearly dictates otherwise. For example, the term “a compound” or “at least one compound” may include a plurality of compounds, including mixtures thereof.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “active ingredient” refers to a pharmaceutical agent including any natural or synthetic chemical substance that subsequent to its application has, at the very least, at least one desired pharmaceutical or therapeutic effect.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1 shows the effects of MB in improving mitochondrial respiration and attenuation ROS production by rerouting electron transfer. (A): Effects of MB on cellular oxygen consumption in HT-22 cells. Cellular oxygen consumption was monitored with sequential injection of MB/Vehicle, oligomycin, FCCP, and Rotenone. The vehicle and MB treated groups had significant difference in oxygen consumption rates (OCR) at all time points (n=5). (B-D): OCR (mitochondrial respiration) and extracellular media acidification rates (ECAR, cellular glycolysis) were monitored with similar sequential treatment in (A) after four hour MB/vehicle incubation at the indicated concentration. MB induced a significant dose-dependent increase in OCAR, a dose dependent decrease in ECAR, and a dose dependent attenuation of rotenone induced OCR inhibition (n=5);

FIG. 2 shows the dose-dependent effects of MB in mitochondrial complex I-III and II-III activity in mitochondrial extracts. A: representative assay of complex I-III activity. B: Complex I-III activity with increasing dose of MB, and N-acetyl MB whose redox action is disabled. C: Complex I-III activity with increasing dose of MB in the presence of complex III inhibitor (Antimycin A). D: Complex I-III activity with increasing dose of MB with and without the complex I inhibitor (rotenone). * and *** indicate significant difference from control groups without MB;

FIG. 3 shows the effects of MB in rotenone and antimycin A induced mitochondrial dysfunction and cell death. A-B: Neuroprotective effect of MB on rotenone (A) and antimycin A (B) induced cytotoxicity in HT22 cells. ** indicates significant difference from vehicle. C: MB has no protection against H₂O₂ (released by glucose oxidase) induced cytotoxicity. D-E: Effects of MB on rotenone induced mitochondrial specific superoxide (D) and total cellular ROS (E). Y axis is percent of max cell numbers, X is intensity. Shown is the representative result from three independent sets of experiments;

FIG. 4 shows the effects of MB on rotenone induced neurological and behavioral deficit in rats. A: MB prevents rat body weight loss induced by rotenone treatment. B: MB improved coordinated motor function deficiency induced by chronic rotenone treatment. ** indicates significant difference between indicated groups (P<0.01). * indicates that there is significant difference between ROT/Sal and both of the other two groups in the individual sessions (P<0.05). C: Effects of MB on randomized blinded neurological assessments. *** indicates significant difference between indicated groups (P<0.001). D-E: Effects of MB on catalepsy measurements in rotenone and MB treated rats with bar test (D) and grid test (E): F: Effects of MB on stride length in rotenone and MB treated rats in gait analysis. ROT: rotenone treated; Sal: Saline (Vehicle for MB); Veh: Vehicle for rotenone. * and ** indicates significant difference between the indicated groups in panel D-F;

FIG. 5 shows the effects of MB and rotenone on Dopamine and L-Dopac levels, mitochondrial complex I-III activity, and total ROS in vivo. A-D: Effects of rotenone and MB treatment in striatum dopamine (A) and L-Dopac (B) levels. C-D: Mitochondrial I-III activity (C) and total ROS (D) in rotenone and MB treated rats. All numbers were normalized to percent of control groups. * and ** indicates significant difference between indicated groups in all panels;

FIG. 6 illustrates the proposed mechanism by which MB re-routes electrons transfer in the ETC in the presence of rotenone and antimycin A inhibition;

FIG. 7 shows Complex II-III activity with increasing dose of MB with and without the complex III inhibitor, antimycin A (AA). Upper panel: a representative complex II-III activity assay shows the no effects of MB on overall complex II-III activity. Lower panel: quantitative analysis result shows the no effects of MB on complex II-III activity in the presence of AA. Enzymatic activity was calculated as the maximal slop of each curve as showed in the upper panel;

FIG. 8 shows the effects of Rotenone and MB on 5HT levels in striatum. No significant difference was found between each group. ROT: rotenone treated; Sal: Saline (Vehicle for MB); Veh: Vehicle for rotenone;

FIG. 9 shows the structure of methylene blue and its derivatives.

FIG. 10 shows the effect of methylene blue and its derivatives on cell viability against glutamate toxicity in HT-22 cells, showing that a side chain at N-motif (Chlorpromazine, promethazine, imipramine) significantly decrease the potency and efficacy of the protective action. In addition, the S-motif is also critical as replacing S to N (Neutral Red) significantly decrease the potency and efficacy of the protection

FIG. 11 shows the effect of methylene blue and its derivatives on reactive oxygen species production induced by glutamate in HT-22 cells, showing that a side chain at N-motif (Chlorpromazine, promethazine, imipramine) significantly decrease the potency and efficacy of the protective action. In addition, the S-motif is also critical as replacing S to N (Neutral Red) significantly decrease the potency and efficacy of the protection.

FIG. 12 shows the effect of MB and its derivatives on mitochondrial membrane potential collapse induced by glutamate in HT-22 cells, showing that a side chain at N-motif (Chlorpromazine, promethazine, imipramine) significantly decrease the potency and efficacy of the protective action. In addition, the S-motif is also critical as replacing S to N (Neutral Red) significantly decrease the potency and efficacy of the protection.

### DETAILED DESCRIPTION OF EXEMPLARY EMBODIMENTS

Mitochondria are the power houses and the major source of free radicals in almost all cells. Mitochondrial dysfunction is implicated in numerous neuropathological diseases, including Parkinson's disease (PD), Alzheimer's disease (AD) and stroke. In addition to ATP production, mitochondria participate in diverse cell signaling events and are essential organelles for cell survival. The oxidative phosphorylation machinery is composed of five complexes (complex I-V). From Krebs cycle intermediates (NADH and FADH₂), electrons feed into complex I or II, and are transferred to complex III, then to complex IV, and finally to O₂. Energy released during the electron transfer is utilized to actively pump out H⁺ from the mitochondrial matrix to the intermembrane space, generating the electrochemical gradient of H⁺ across the inner membrane, which is ultimately utilized by complex V to produce ATP. However, a small portion of electrons leaking from the electron transport chain (ETC), mostly at complex I and complex III, react with molecular oxygen and yield superoxide anion, which can be converted into other reactive oxygen species. When ROS production overwhelms the endogenous antioxidant systems, they can potentially damage various cellular components, including proteins, lipids, and nucleic acids. ROS is implicated in aging and various pathological processes, and has been proposed as the key culprit for many neurodegenerative diseases. Studies have shown that it is not the mitochondrial energy defect per se, but rather the over production of ROS induced by blockage of mitochondrial complexes that accounts for the neurodegeneration in many neuropathological conditions. Thus, mitochondrial targeting strategies such as free radical scavengers, mitochondrial signaling regulation, and electron transfer chain (ETC) component supplementation have been extensively studied. Disappointingly, none of these neuroprotective strategies has been proven successful in any neurological diseases in clinical trials.

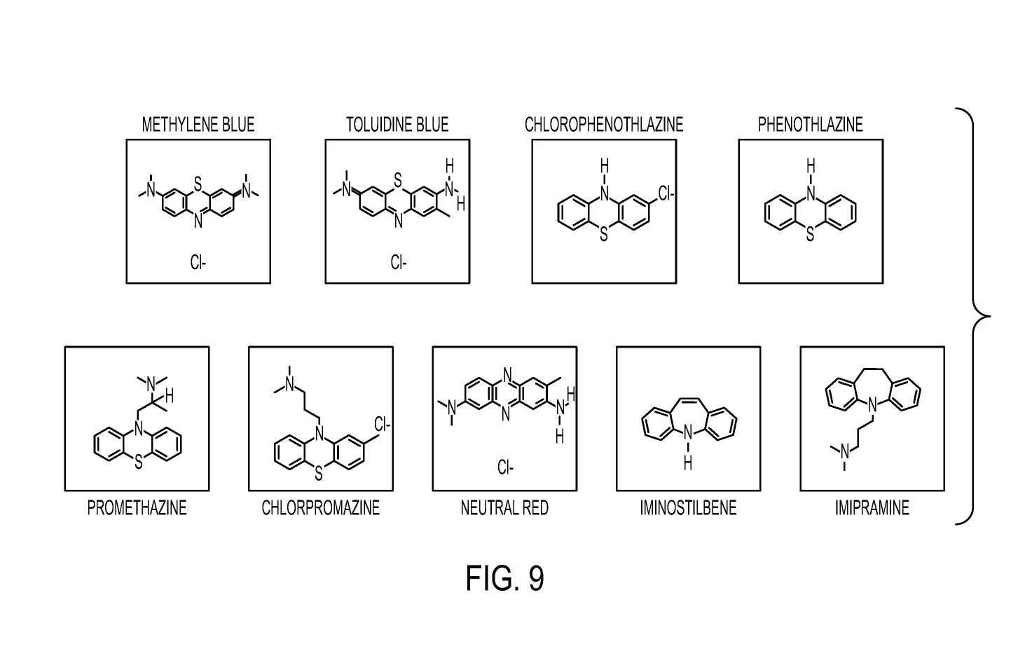

Methylene blue (MB) is a heterocyclic aromatic compound (FIG. 9) that has many biological and medical applications. It is an FDA approved drug for methemoglobinemia and an antidote to cyanide poisoning. Previous publications have suggested that MB improves mitochondrial function. In an embodiment of the invention, MB and similar compounds are used as an alternative electron carrier that efficiently shuttle electrons between NADH and cytochrome c (cyt c). This process reroutes electron transfer upon complex I and III inhibition, reduces electron leakage, and attenuates ROS over-production. MB is protective in a rotenone induced animal model of Parkinsonism and an animal model of ischemic stroke induced by middle cerebral artery (MCA) occlusion.

The present invention is directed to compositions comprising MB and similar compounds, such as leuco-methylene blue and acetyl-methylene blue, having a beneficial therapeutic activity and uses thereof. The present invention is further directed to methods of uses of the compositions as antioxidants and/or neuroprotective agents for the treatment of medical conditions associated with oxidative stress and/or neural damage, such as, for example, neurological diseases, disorders and trauma, and hence in the treatment of CNS-associated diseases, disorders and trauma.

The principles and operation of the present invention may be better understood with reference to the figures and accompanying descriptions.

Before explaining the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As is discussed hereinabove, the central nervous system (CNS), governing all function of a living organism, from autonomous functions such as breathing, bowel movements and reflexes to cognitive capacities such as learning, memory and other mental functions, is a highly complex system which is sensitive to any electrical and chemical imbalance. These imbalances are often expressed in what is referred to herein as neurodegenerative diseases and/or CNS-associated diseases, disorders or trauma, causing symptoms which range from mild discomfort to complete impairment and death.

Oxidative stress, caused by reactive oxygen species, represents another injury mechanism implicated in many of the same acute and chronic diseases and conditions. Reactive oxygen species, e.g., superoxide radicals, would cause oxidative damage to cellular components, such as peroxidation of cell membrane lipids, inactivation of transport proteins, and inhibition of energy production by mitochondria.

### WORKING EXAMPLES

### Animals and Reagents:

Male Sprague-Dawley rats (230-250 g) (Charles River, Wilmington, Mass.) were housed individually with controlled temperature (22-25° C.) and humidity (55%). A 12-hour light-dark cycle was maintained with lights on between 7 a.m. and 7 p.m. All housing and procedures were performed in accordance with the guidelines of the Institutional Animal Care and Use Committee of the National Research Council, and were approved by the University of North Texas Health Science Center Animal Care and Use Committee. MB was purchased from Akorn Pharmaceutics (Lake Forest, Ill.). N-Acetyl MB was synthesized by Omm Scientific, Inc (Dallas, Tex.).

### Methylene Blue-Containing Compositions:

MB is widely used as a redox indicator in analytical chemistry. At pH 7.0 the MB has a very low redox potential of 11 mV and is very efficient cycling between oxidized and reduced forms. It has been demonstrated that MB could oxidized NADH and transfers a pair of electrons from NADH to molecular oxygen in a closed system and that MBH2 prefer to directly reduce cyt c even in the presence of oxygen. These chemistry data suggest that MB might be able to function as an electron carrier in the ETC.

Methylene blue is readily soluble in water (1 gm/25 ml) and therefore can be solubilized in physiologic saline, which was used in the animal studies described herein. This high aqueous solubility allows its formulation in a variety of pharmaceutically acceptable carriers, including tablets, capsules suppositories, transdermal patches, intramuscular and subcutaneous injection solutions and intravenous solutions.

### Mitochondrial Isolation and Mitochondrial Respiration Chain Activity Assays:

Fresh rat heart was removed and placed in ice-cold mitochondrial isolation buffer containing 210 mM mannitol, 70 mM sucrose, 5 mM HEPES, and 1 mM EDTA, pH 7. The heart was homogenized immediately, and the mitochondrial fraction was isolated by differential centrifugation as described previously (Trounce et al., 1996). Mitochondria were stored at −80° C. until subsequent analysis.

All mitochondrial activity assays were performed according to previous publications with minimal modifications for complex I (Lenaz et al., 2004), complex I-III, complex II, complex III, and II-III (Trounce et al., 1996). All assays used a similar master mix that contained potassium phosphate pH 7.4 (50 mM), BSA 2.5mg/ml, MgCl₂ 5 mM, KCN (2 mM), and various inhibitors or substrates, including rotenone (6 μM), antimycin A (2 μM), NADH (150 μM), CoQ1 (60 μM), cyt c (50 μM), succinate (8 mM), and DBH₂ (100 μM). Complex I activity was monitored by adding NADH to the mix with or without rotenone/antimycin A. The final data were normalized to blank values at time zero. The reaction was monitored in a kinetic spectrophotometer at 340 nm. Complex I activity was measured in arbitrary units and normalized to control levels for statistical analysis. Isolated mitochondria equivalent to 40-200 μg mitochondrial protein was used for each assay, except for assays with inhibitors, when larger amount of mitochondria (3 to 5 folds) were used to ensure that inhibition was distinguishable. Final data were normalized to control levels. For complex I-III activity, the reaction was initiated with the addition of NADH, cyt c was used as final substrate and its reduction was monitored at 550 nm. For assay of complex II-III activity, succinate was added instead of NADH, and the electron transfer to cyt c was performed according to previous publications (Atamna et al., 2008). To test whether the electrons from the reduced form of MB (MBH₂) can be transferred to cyt c in mitochondrial lysate, we added 10 μM 2,7-dichlorofluorescin (DCFH, Invitrogen, Carlsbad, Calif.) to the reaction system. Free radicals convert non-fluorescent DCFH to highly fluorescent DCF (Ex, 488 nm, and Em, 530 nm). In the presence of cyt c, which accepts electrons from MBH₂, conversion of DCFH to DCF is decreased.

### Cell Viability Assay:

HT-22 cells (gift from Dr. David Schubert, Salk Institute, San Diego, Calif.) were plated at 4,000 cells per well in 96 well plates and cultured over night. Then cells were treated with vehicle (DMSO), rotenone (2-8 μM), antimycin A (1-10 μg/ml), or glucose oxidase (2-10 mU) in the presence or absence of various concentrations of MB for 24 hours. At the end of the 24 hours, the medium was removed and the plates were rinsed with PBS and incubated with 10 μM calcein-AM (Invitrogen, Carlsbad, Calif.) in PBS for 20 minutes. Fluorescence was determined using a TECAN M200 microplate reader (San Jose, Calif.) with an excitation/emission set at 485/530 nm. Cell culture wells treated with bleach before rinsing were used as blank.

### Seahorse XF-24 Metabolic Flux Analysis.

HT22 cells were plated at 8,000/well and cultured on Seahorse XF-24 plates. Cells were grown in previously described medium and environments for 24 hr. On the day of metabolic flux analysis, cells were changed to unbuffered DMEM (DMEM base medium supplemented with 25 mM glucose, 10 mM sodium pyruvate, 31 mM NaCl, 2 mM GlutaMax, pH 7.4) and incubated at 37° C. in a non-CO2 incubator for 1 hr. All medium and injection reagents were adjusted to pH 7.4 on the day of assay. Four baseline measurements of oxygen consumption rate (OCR) and extracellular acidification rate (ECAR) were taken before sequential injection of mitochondrial inhibitors. Specific readings indicated by the arrows were taken after each addition of mitochondrial inhibitor before injection of the subsequent inhibitors. The mitochondrial inhibitors used were oligomycin (10 μM), FCCP (1 μM), and rotenone (5 μM). OCR and ECAR were automatically calculated and recorded by the Seahorse XF-24 software. After the assays, plates were saved and protein readings were measured for each well to confirm equal cell numbers per well. The percentage of change compared with the basal rates was calculated as the value of change divided by the average value of baseline readings.

### Mitochondrial Superoxide and Cellular ROS Analysis:

Mitochondrial specific superoxide and cellular ROS were measured with specific fluorescent probes. Briefly, HT-22 cells were treated with vehicle or 2 μM rotenone, with or without 100 ng/ml MB for 4 hr. The cells were dissociated, then incubated in pre-warmed PBS containing 0.5% BSA and H2DCFDA (Cellular ROS) or MitoSox (mitochondrial superoxide) for 15 min at 37° C., followed by washing with PBS. The cells were then resuspended in supplemented RPMI-1640 for further incubation. The cells were analyzed with FITC channel (Cellular ROS), or PE channel (MitoSox) using a BD™ LSRII flow cytometer.

### Effect of MB on a Rotenone Model of PD:

Rats were assigned to three groups: control animals received vehicle infusion and daily saline injection (Veh/Sal N=6-8); a second group received rotenone infusion at the dose of 5 mg/kg/day and daily saline injection (ROT/Saline N=8-11); and the third group received rotenone infusion (5 mg/kg/day) and MB injection at the dose of 500 μg/kg/day (ROT/MB N=8-10). Vehicle or rotenone was infused via Alzet osmotic mini pumps that were filled with vehicle or rotenone dissolved in vehicle (equal volumes of dimethylsulfoxide and polyethylene glycol). Pumps containing vehicle or rotenone were sterilized with UV irradiation and implanted subcutaneously on the back. Intraperitoneal injections were administered with normal saline or MB, 500 μg/kg/day, diluted in normal saline to 500 μl of final volume. Rats were sacrificed on day 8 at four hours after last dose of MB or saline treatment.

### Accelerating Rotarod Performance:

The accelerating rotarod, a motor driven treadmill (Omnitech Electronics, Columbus, Ohio) measures running coordination and motor performance. The rotor consisted of a nylon cylinder with a 7 cm diameter mounted horizontally 35.5 cm above a padded surface. On a given trial, the rats were placed on the cylinder, the cylinder was rotated, and a timer switch was simultaneously activated. Acceleration continued from 0 to a maximum of 44 rpm until animals were unable to stay on top of the rotating rod and fell to the padded surface. Animals were removed at 90 seconds if they did not fall. When the rat landed on the surface, a photosensitive switch was tripped and the timer stopped. Rats were subjected to this test two sessions a day, three trials per session, for 7 sessions. A 20-minute resting period was given between each trial. Animals were given at least two hours break time between sessions each day. Retention times on the rotating treadmill were recorded and plotted for analysis.

### Neurological Assessment:

Neurological assessment was performed by three evaluators blind to treatment conditions. The evaluators were initially trained until they produced consistent scores when evaluating rats in these tests. Subsequently, experimental subjects were randomized and presented to the evaluators individually. For each session, which consisted of three trials, an individual evaluator scored a rat in only one trial; thus, the final score for a session for each animal was the average score of the three evaluators. The category included: tremor; locomotion; bradykinesia; hypokinesia; posture; and gross motor skills. The details on the scoring systems were adapted from previous publications in various animal models including MPTP and 6-OHDA models. Specific rating scales are provided below:

### Neurological Assessment Scoring System:

### Tremor:

0 Absent
1 Occasional or barely detectable (normal for aged animals), occurring while active
2 Frequent or easily detectable, occurring while active or at rest
3 Continuous or intense, occurring while active or at rest

### Locomotion

0 Uses all four limbs smoothly and symmetrically
1 Walks slowly (normal for aged), noticeable limp
2 Walks very slowly and with effort, may drag limb.
3 Unable to ambulate

### Bradykinesia

0 Quick, precise movements
1 Mild slowing of movements (normal for aged)
2 Slow deliberate movements with marked impairment initiating movements
3 No movements

### Hypokinesia

0 Moves freely, alert, responsive
1 Reduced activity (normal for aged), moves less frequently (without provocation)
2 Minimal activity, moves with provocation.
3 Akinetic (essentially no movements)

### Posture

0 Normal posture, able to stand freely
1 Reduced posture (normal for aged), stands with limbs apart,
2 Stooped posture, hunched, legs bent, often lean on cage walls
3 Unable to maintain posture, recumbent

### Gross Motor Skills

0 Normal function, able to retrieve small objects accurately
1 Reduced ability/frequency of retrieving small objects
2 Great difficulty in retrieving small objects, rarely used
3 Unable to retrieve objects

### Catalepsy Measurement:

Catalepsy was measured by standard bar test and grid test adapted from previous publication. For bar test, the rats were placed with both front paws grasp on a horizontal bar which was 9 cm above the surface. The time, each animal took to remove one paw from the bar was manually measured by a stopwatch. For grid test, rats were placed on a vertical wire grid (25.5 cm width and 44 cm high with a space of 1 cm between each wire) and catalepsy was determined by the length of time the animals maintained all four paws on the grid using a stopwatch. The maximum descent latency was set at 300 seconds for the bar test and 60 seconds for grid test.

### Gait Analysis for Stride Length:

For gait analysis, the fore and hind paws of each rat were painted with blue and red dye, respectively. The animals were allowed to walk across an absorbent paper covered straight alley runway (150 cm long, 20 cm wide, opaque walls 20 cm tall) into a dark compartment. Before the test, animals were put in the dark compartment for 2 minutes to habituate to the environment and 3 pre-training runs were conducted for all rats. Three trials were conducted to obtain clear footprints for each animal. Prolonged stop or turning backward in the runway was considered failed trials. The distance of individual steps were analyzed by two independent blinded observers. Average of the left and right stride distance for each animal was used for statistical analysis.

### Tissue Collection:

After completion of neurological assessments and behavioral tests, rats were killed on the 8^{th} day of rotenone infusion under anesthesia induced by xylazine (20 mg/kg, i.p.) and ketamine (100 mg/kg, i.p.). All osmotic pumps were checked for the residual liquid to ensure proper drug delivery. One hemisphere was fixed by 4% paraformaldehyde in phosphate buffer for 48 hr until processing for sectioning. The other hemisphere was snap-frozen in liquid nitrogen, and kept at −80° C. until extraction for biochemical analysis. Striatum dissected from a separate set of animals were used for analysis of monoamines by HPLC.

### HPLC Analysis of Monoamines and Metabolites:

For monoamines and metabolites measurement, striatum tissue was dissected from each hemisphere, weighed, and stored at −80° C. For HPLC analysis, tissue samples were sonicated in 9 volumes of 0.1 M perchloric acid containing 0.2 mM sodium metabisulfite and centrifuged at 15,000 rpm for 20 minutes 4° C. in a benchtop centrifuge to clear debris. Five μL of cleared supernatant was injected onto a C18 HPLC column and separated by isocratic elution at a flow rate of 0.6 ml/min with MD-TM mobile phase (ESA Inc, Chelmsford, Mass.). Neurotransmitter monoamines and metabolites were detected using an ESA CoulArray electrochemical detector with a model 5014B cell set to a potential of +220 mV. Peak areas were compared to a standard curve of external standards to calculate quantities of dopamine and metabolites per mg tissue.

### Immunohistochemistry:

Rat hemispheres were sequentially incubated in 10%, 20% and 30% sucrose over night. The brains were then frozen in optimal cutting temperature) compound (VWR, San Francisco Calif.) and sectioned in the coronal plane with a cryostat to produce 20 μm floating sections. For single immunohistochemistry studies, 20-μm sections were blocked with 5% normal goat serum and incubated in primary antibodies at 4° C. over night. Sections were then immunostained with Picture Plus immunohistochemistry kits (Invitrogen, Carlsbad, Calif.). 3,3′-diaminobenzidine tetrachloride (DAB) was used to visualize the sections. The primary antibodies used include a monoclonal mouse antibody against tyrosine hydroxylase (TH) (1:500) (Millipore, Billerica, Mass.), a monoclonal anti-ubiquitin (1:800) (Millipore, Billerica, Mass.), and a monoclonal anti-α-synuclein (1:100) (Santa Cruz, Santa Cruz Calif.). For fluoro-jade B staining, paraformaldehyde-fixed brain sections from brains in each group were stained according to the manufacturer's protocol (Millipore Billerica, Mass.). The slides were first immersed in 1% NaOH in 80% alcohol for 5 minutes, followed by washing for 2 minutes in 70% alcohol and 2 minutes in distilled water. The slides were then transferred to a solution of 0.06% potassium permanganate for 10 minutes to ensure consistent background suppression between sections. The slides were then stained with 0.0004% fluoro-jade B solution for 20 minutes. Double labeling for TH and fluoro-jade B was achieved by immunofluorescence. Sections were initially stained with fluoro-jade B, then, incubated with antibodies against TH (1:500; Chemicon) for 1 hr at room temperature, and Alexa 594 conjugated goat anti-mouse IgG was used to detect TH-positive cells. For controls, primary antibodies were omitted. Double stained sections were examined with conventional fluorescence microscopy, and images were captured on a Zeiss microscope linked to an image analysis system with selective filter sets to visualize FITC, rhodamine, and DAPI separately. DAB immunostained sections were visualized with bright-field microscope, and images were collected with a colored digital camera.

### Total ROS Assay:

Frozen brain tissue was placed in 10-fold volume/weight ice cold PBS containing a protease inhibitor cocktail (EMD bioscience Gibbstown, N.J.) and homogenized rapidly with a polytron power homogenizer. An aliquot was ultra-centrifuged at 50,000 g for 30 min. Supernatant was removed and analyzed for total ROS content using the fluorescence probe DCFH. DCFH was dissolved in absolute ethanol and diluted with PBS (pH 7.4) to 125 μM, and added to sample brain extracts at a final concentration of 25 μM followed by incubation at 37° C. for 10 minutes. Fluorescence intensity was measured using a fluorometer (TECAN M200 microplate reader San Jose, Calif.) at an excitation/emission wavelength set at 485/530 nm. Data were obtained as relative fluorescent intensity (RFI) and normalized to the percent of control. DCFH without brain extracts was used as blank and was consistently less than 1% of control group.

### Transient Focal Cerebral Ischemia:

Transient focal cerebral ischemia was induced by intraluminal filament MCA occlusion (Liu et al., 2005). Briefly, the left internal carotid artery (ICA) was exposed, and a 3-0 monofilament nylon suture was introduced into the ICA lumen through a puncture and gently advanced to the distal ICA until proper resistance was felt. After 1 hr, the suture was withdrawn for reperfusion. At 24 hr after reperfusion, the animals were sacrificed and the brain harvested. The brains were sectioned at 3, 5, 7, 9, 11, 13 and 15 mm posterior to the olfactory bulb. Each slice was incubated 30 min in 2% 2,3,5-triphenyltetrazolium chloride (TTC) in physiological saline, then fixed in 10% formalin. The stained slices were digitally photographed for measurement of ischemic lesion volume (Image-Pro Plus 4.1).

### Statistical Analysis:

The data for mitochondrial complex activity with MB alone, animal body weight, dopamine/5-HT levels and ROS in vivo were analyzed with one-way ANOVA. The data for mitochondrial I-III activity assay with inhibitors, complex II-III assay with inhibitors, and cell viability assays were analyzed by two-way ANOVA (treatment group with concentration). Rotarod assay and neurological assessment were analyzed by three-way ANOVA (group, session/category, and performance). When a significant difference was detected by ANOVA, a post hoc Tukey's test was performed to identify a specific difference between groups. Values were expressed as mean±standard error of mean (SEM). Between two indicated samples, student-t-tests were used to acquire P value. The p value<0.05 was used to indicate statistical significance. In all assays * indicates (P<0.05), ** indicates (P<0.01), and *** indicates (P<0.001).

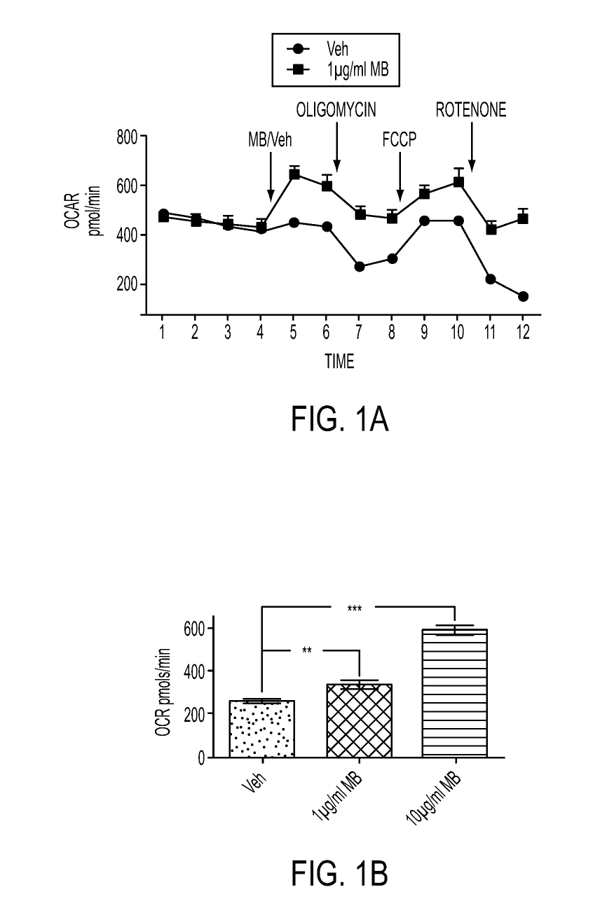

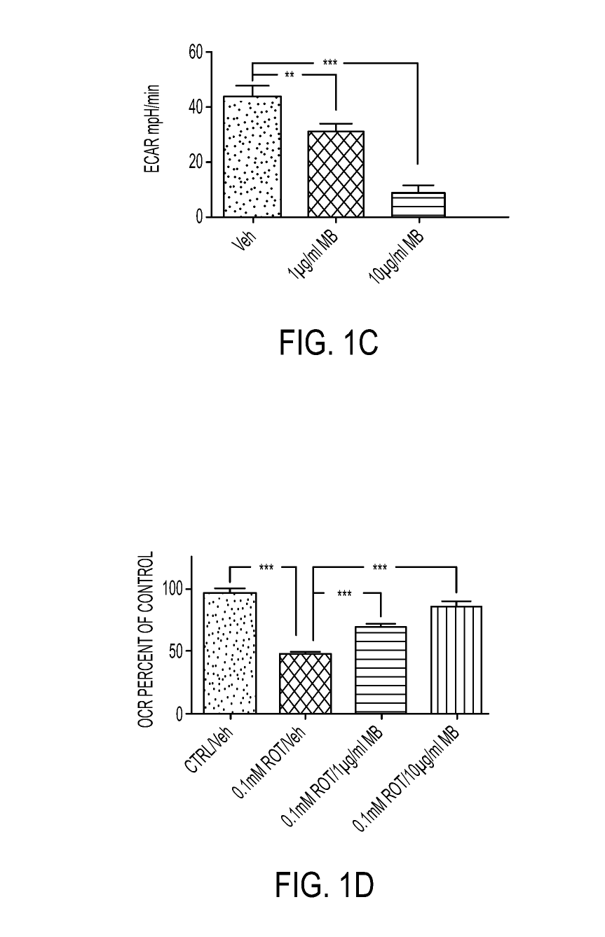

In an embodiment of the invention, a composition comprising MB or similar compounds is used to provide therapeutic benefit by increasing oxygen consumption rates. In another embodiment of the invention, a composition comprising MB or similar compounds is used to provide therapeutic benefit by reducing glycolysis. MB has been previously reported to improve mitochondrial functions. In our initial experiments, we used Seahorse Cellular Bioenergetic analyzer to examine the effects of MB on mitochondrial respiration and cellular glycolysis in transformed hippocampal HT-22 cells. Oligomycin (complex V inhibitor), FCCP (uncoupler), and rotenone (complex I inhibitor) was sequentially added to demonstrate specific action sites in the ETC. MB rapidly increased OCR and maintained enhanced functions with all treatments (FIG. 1A). With two hour incubation, MB dose-dependently increased OCR from 264±10 pmol/min with vehicle treatment to 594±19 pmol/min with 10 μg/ml MB treatment (FIG. 1B), and reduced ECAR from 44±4 mPH/min with vehicle treatment to 9±3 mPH/min with 10 μg/ml MB treatment (FIG. 1C). In addition, MB dose-dependently attenuated rotenone induced OCR inhibition (FIG. 1D).

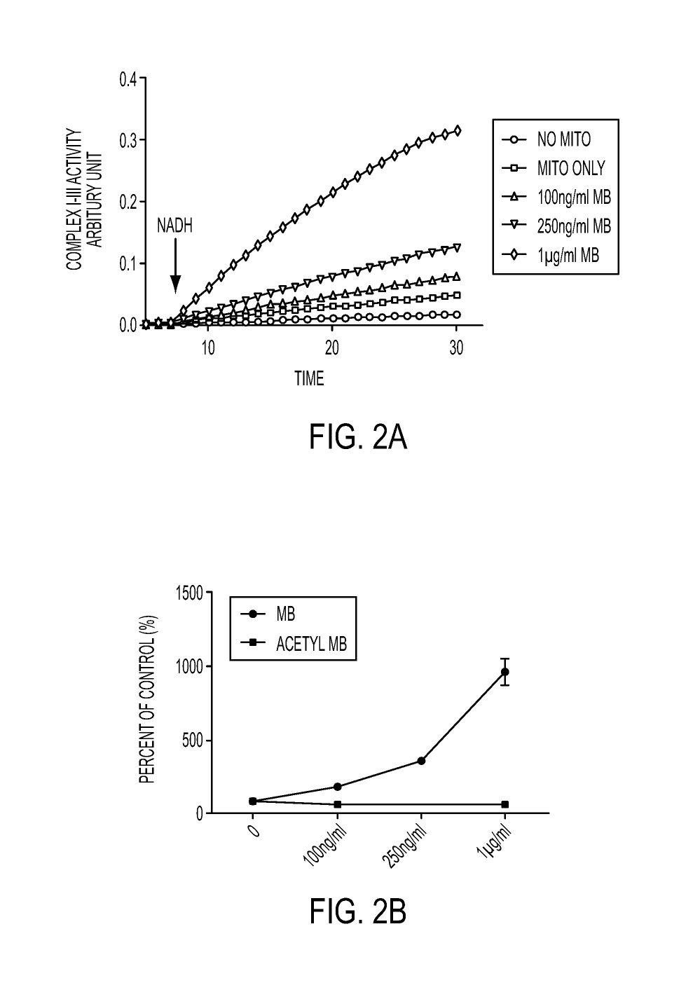

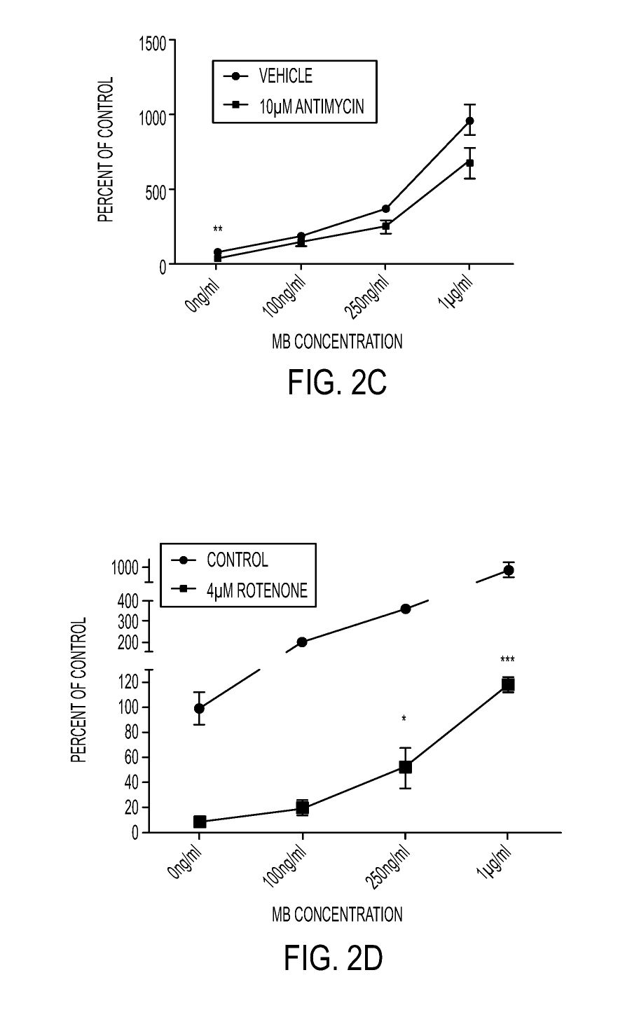

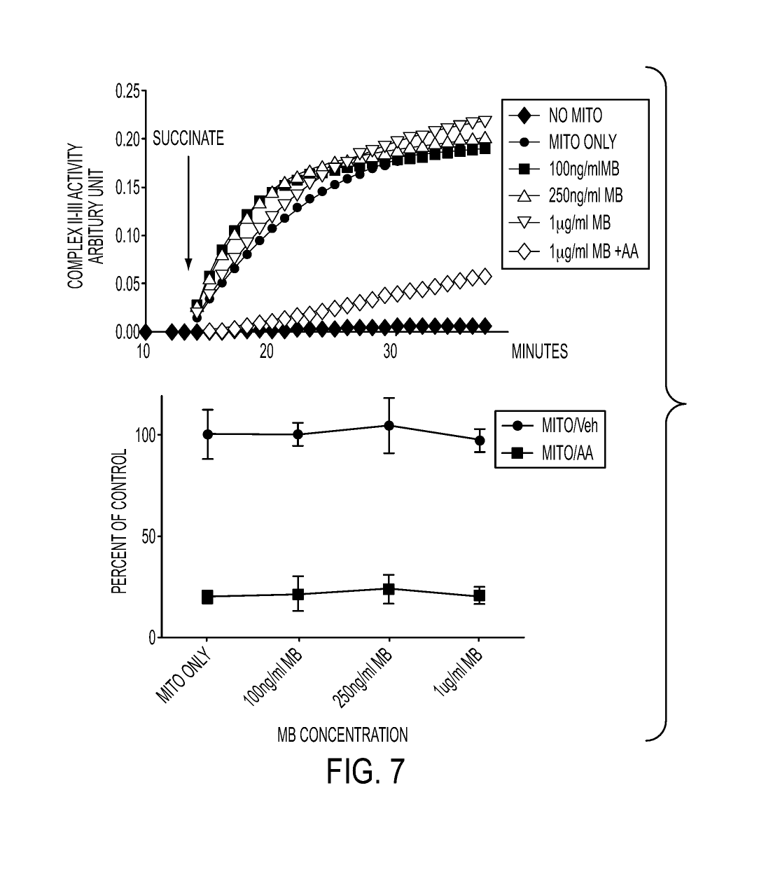

In another embodiment of the invention, a composition comprising MB or similar compounds is used to provide therapeutic benefit by increasing complex I-III activity. The effects of MB on the overall activity of mitochondrial ETC complex I-III was analyzed in extracted mitochondria. MB dose-dependently enhanced mitochondrial complex I-III activity, as evidenced by the enhanced electrons transfer from NADH to cyt c. An up to nine-fold increase of complex I-III activity was detected in the presence of increasing doses of MB (0.1-1 μg/ml) (FIG. 2A-B). As a proof-of-concept for the mechanism, MB was modified with N-acetylation. This acetylation blocked the redox capacity of MB, and completely eliminated the action of MB on complex I-III. Similar assays were performed in the presence of a specific complex III inhibitor (antimycin A) (FIG. 2C) and a specific complex I inhibitor (rotenone) (FIG. 2D). A 90% and 70% inhibition of complex I-III activity was induced by rotenone and antimycin A, respectively. MB treatment dose-dependently prevented the inhibitory action of antimycin A and rotenone. Specifically, 1 μg/ml MB increased I-III activity to 674±99% of the control level in the presence of antimycin A (FIG. 2C). MB (1 μg/ml) increased such activity to 118.1±6.4% of control in the presence of rotenone (FIG. 2D). On the other hand, MB had no significant effects on complex II-III activity, for which 80% of activity was inhibited by antimycin A (FIG. 7). These data demonstrate that MB is actively involved in the electrons transfer process in mitochondria.

In a further embodiment of the invention, a composition comprising MB or similar compounds is used to provide therapeutic benefit by rerouting electrons between complex I and IIII. The effects of MB on individual mitochondrial complex I, II, and III activities were further studied. MB had no significant effect on complex II or complex III activity alone (data not shown). However, it was found that MB was a direct substrate of NADH dehydrogenase in mitochondrial complex I. In a typical complex I activity assay, CoQ1, an endogenous substrate for complex I, was used to receive the electrons from NADH. This enzymatic reaction was very sensitive to rotenone (above 95% inhibition). The results indicate that MB can completely replace CoQ1 as a direct substrate to accept the electrons from NADH, and convert MB to the reduced form (MBH₂). Such activity is relatively insensitive to rotenone inhibition (26.7% at maximum rotenone concentrations). It was also observed that electrons from reduced MB (MBH₂) can be further delivered to cyt c. In this experimental setup, DCFH was used as a probe to monitor electron transfer. Without cyt c, MB facilitated the electron transfer from NADH and enhanced the oxidation of DCFH probe. In the presence of cyt c, MBH₂ transferred electrons to cyt c instead of DCFH, leading to a decrease in DCFH oxidation. The addition of cyt c significantly decreased oxidation of the DCFH in a dose-dependent manner, suggesting that the reduced form of MB (MBH₂) is able to deliver the electrons to cyt c in mitochondria.

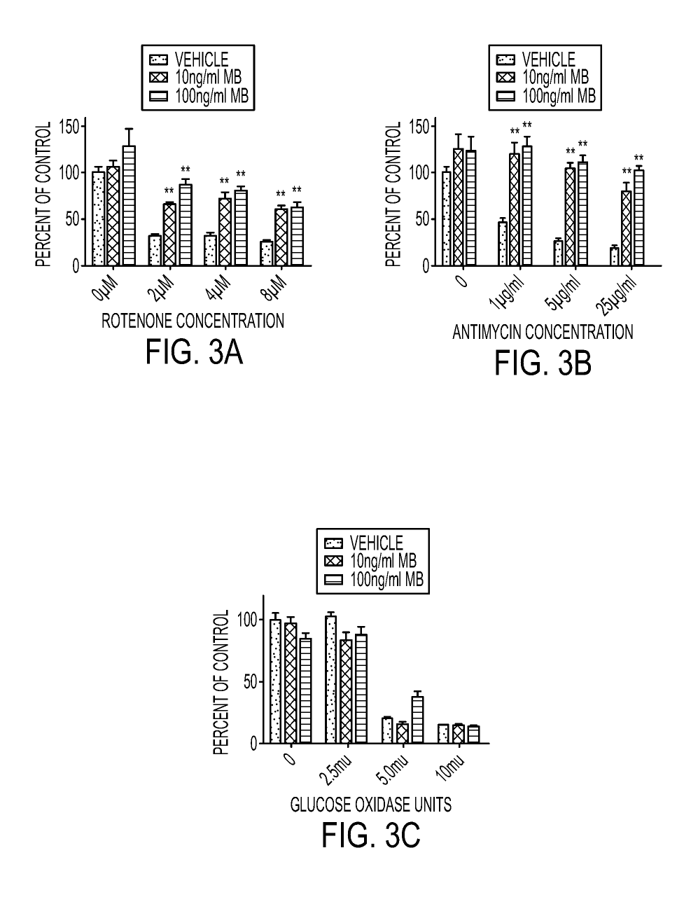

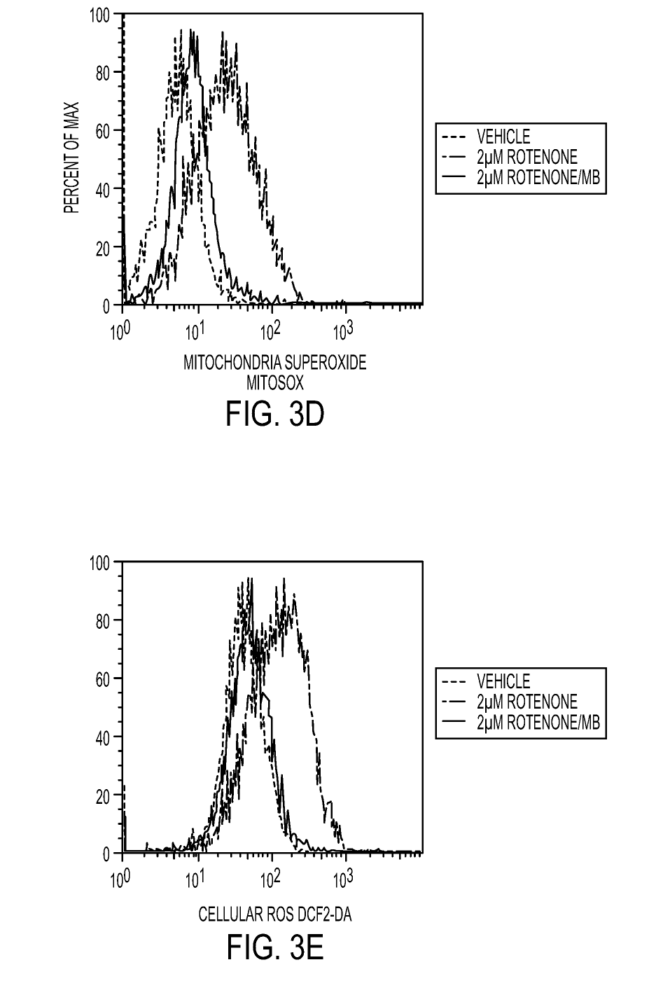

In an embodiment of the invention, a composition comprising MB or similar compounds is used to provide therapeutic benefit against cytotoxicity. The effects of MB treatments on mitochondrial oxidative phosphorylation inhibition in HT-22 cells were examined. A significant protective action of MB against rotenone toxicity (complex I inhibition) was seen at all rotenone concentrations examined. At the highest rotenone concentration (8 μM), cell survival increased from 25.4±1.6% with vehicle co-treatment to 60.5±4.5% with 100 ng/ml MB co-treatment (FIG. 3A). Significant protection was also found with MB against complex III inhibitor (antimycin A) at various concentrations. At the highest antimycin A concentrations tested (25 μg/ml), 19.4±3.0% cells survived without MB, while 80.3±9.6% cell survival was observed with MB at 10 ng/ml (26. 7 nM). A complete protection was found for MB at 100 ng/ml (267 nM) (FIG. 3B).

In cell cultures, MB provides protection against mitochondrial inhibition induced ROS over-production, mitochondrial dysfunction, and cytotoxicity. Given the high enzymatic efficacy and potency, the alternative electron transfer strategy requires much lower concentrations for neuroprotection as comparing with the traditional free radical scavengers. Consistently, the neuroprotective effects of MB occur at very low concentrations with an EC50 of 0.1762 nM against glutamate toxicity in HT22 cells. Therefore, the alternative electron transfer strategy is fundamentally different from traditional free radical scavenger approach. Rather, it avoids the production of ROS by re-routing electron transfer and bypasses complex I/III inhibition. Indeed, MB failed to provide protection against direct hydrogen peroxide insult even with much higher concentrations.

Additionally, it was examined whether MB could function as a direct ROS scavenger. Glucose oxidase chronically oxidizes glucose in the medium to release H₂O₂, which results in cell death. MB failed to protect cells with such H₂O₂ induced oxidative damage (FIG. 3C); thus, MB is not a direct ROS scavenger per se. In contrast, when rotenone was used to generate mitochondrial specific superoxide and ROS production in HT-22 cells, MB (100 ng/ml) almost completely blocked the increase of mitochondrial superoxide (MitoSox probe) and total cellular ROS (DCFH₂-DA) (FIGS. 3D-E).

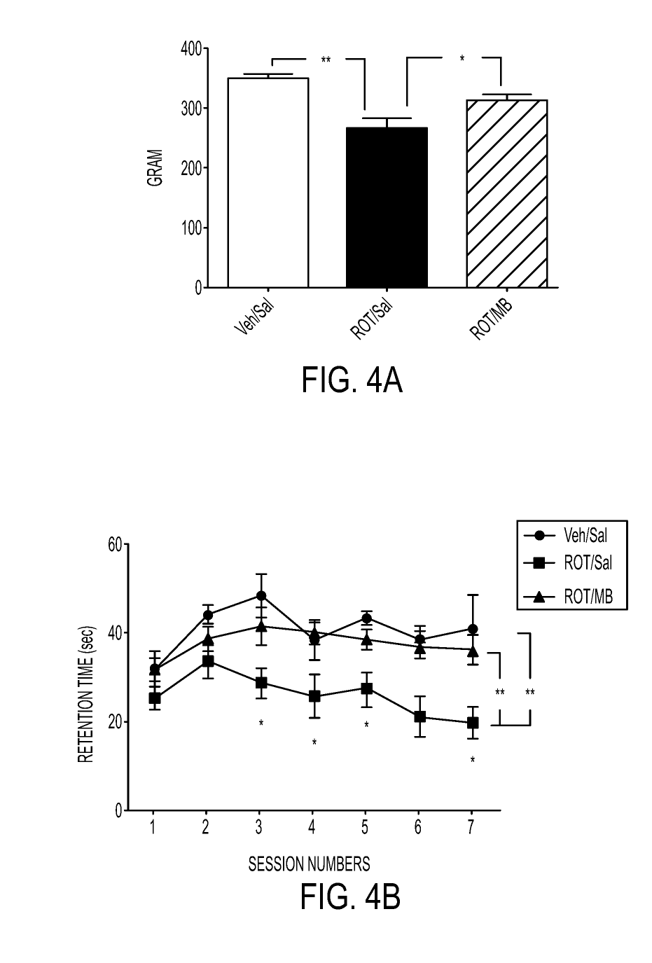

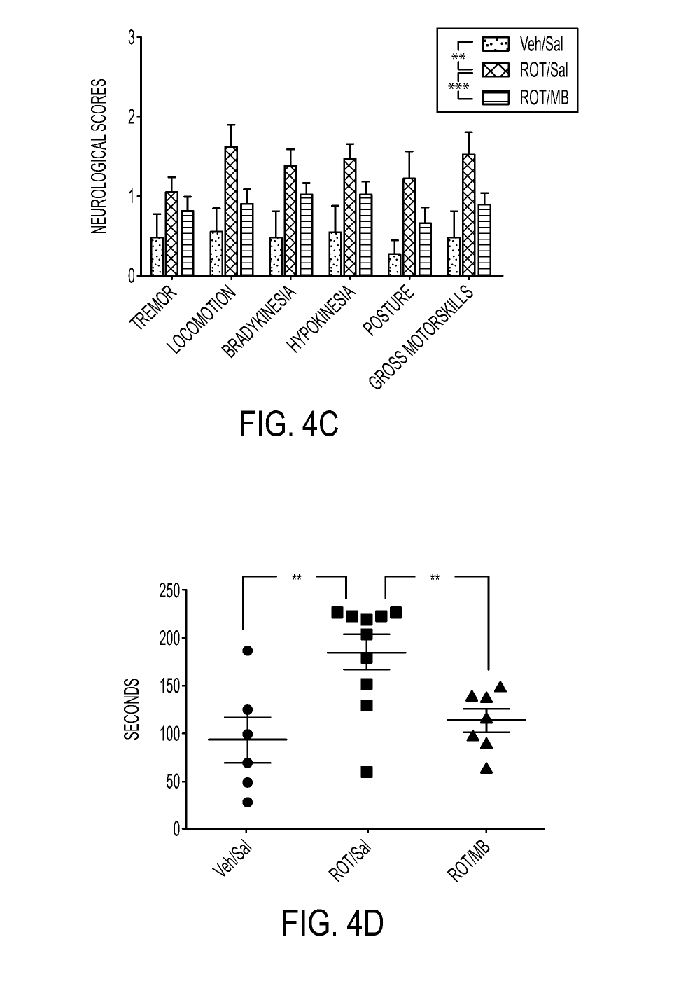

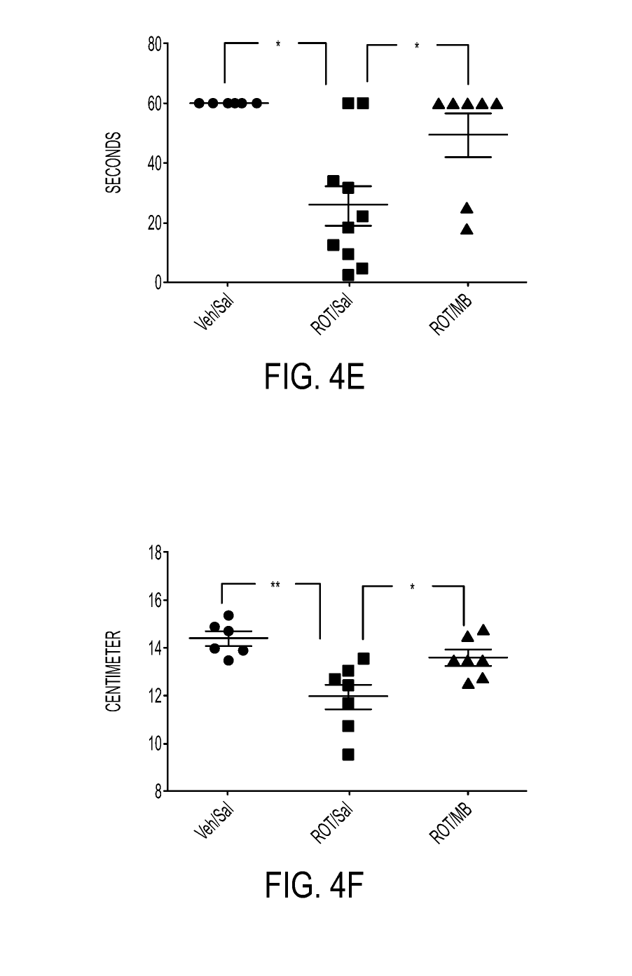

In an embodiment of the invention, a composition comprising MB or similar compounds is used to provide therapeutic benefit by attenuating the effects of a neurodegenerative disease. Systemic rotenone infusion was used to produce a rat model of PD. The effects of MB in this model were determined by neurological and behavioral assessment, biochemical analysis, and neuropathological evaluation. Rats treated with rotenone endured significant weight loss, which was attenuated by MB treatments (FIG. 4A). Significant locomotor deficits were found in rotenone treated rats (FIG. 4B). In rotarod tests, control rats receiving vehicle showed improved performance in early sessions. In contrast, the ROT/Sal group showed decreased performance from the beginning of the sessions (5^{th} day after rotenone infusion), and had lower performance throughout the sessions. Indeed, the improvement of performance seen in controls was replaced by progressive decline of performance in the ROT/Sal group. MB treatment almost completely prevented these rotenone-induced deficits (FIG. 4B). In randomized blinded neurological assessment, significant deficits in all categories were observed in rotenone treated animals, evidenced by the higher score at each assessment. MB treatment significantly improved all neurological scores in rotenone treated rats (FIG. 4C).

Catalepsy was tested by measuring the descent latency after five days of rotenone infusion, using both bar and grid tests. In both tests, rotenone-treated animals showed marked deficiency, which was significantly improved by MB treatment. Specifically, rotenone prolonged descent latency, compared to control animals in bar test, and MB treatment completely reversed such change induced by rotenone (FIG. 4D). Similarly, in grid test, rotenone significantly decreased latency to fall, which was attenuated by MB treatment (FIG. 4E). In gait analysis, rotenone significantly decreased stride length, which was improved by MB treatment (FIG. 4F).

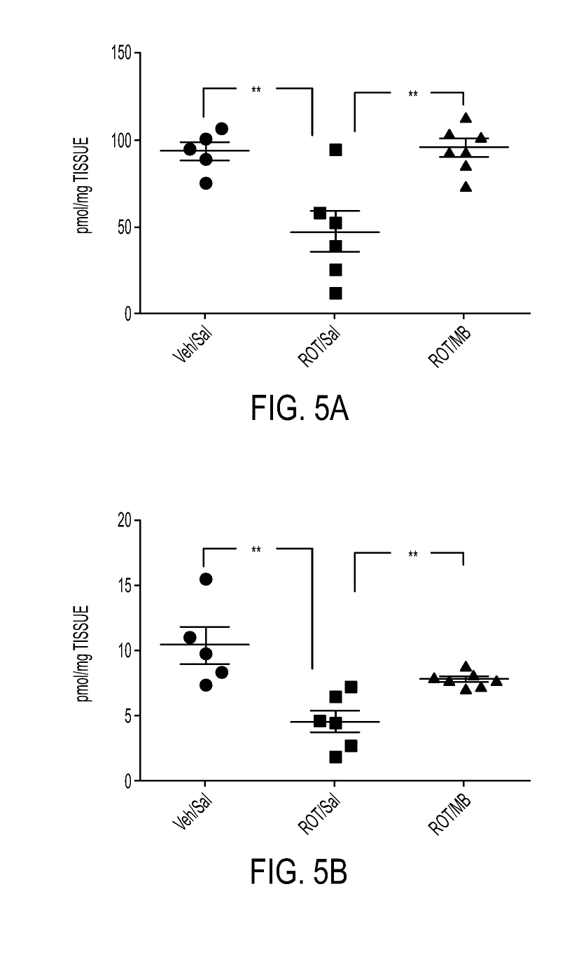

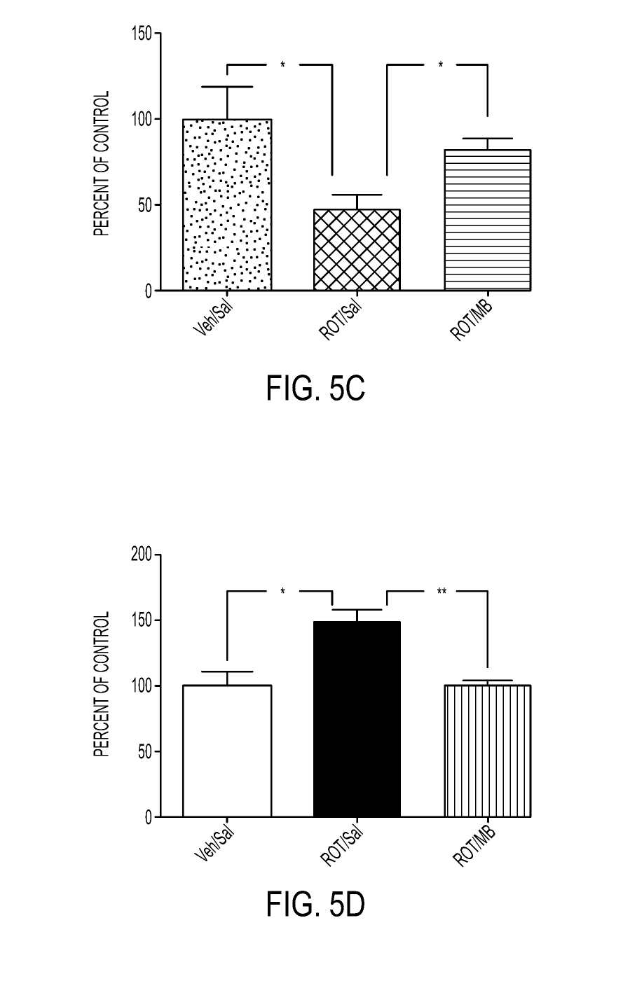

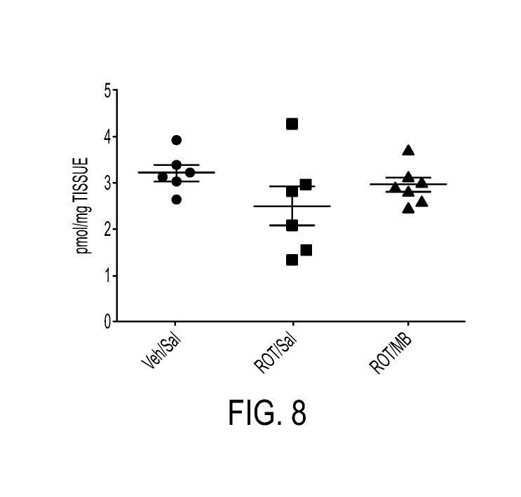

Levels of dopamine, dopamine metabolites, and 5-HT in striatum were analyzed. Dopamine levels (FIG. 5A) as well as its metabolite, 3,4-Dihydroxyphenylacetic acid (L-DOPAC) (FIG. 5B), significantly decreased in striatum derived from rotenone treated group (ROT/Sal), compared with the control group (Veh/Sal). Consistently, such dopamine depletion was almost completely rescued by MB treatment (FIGS. 5A-B). There was no significant differences in 5-HT levels among all three groups of animals (FIG. 8). These results confirmed the selective toxicity in dopaminergic system induced by rotenone. We also assayed the mitochondrial activity in the brain extracts. A significant decrease of complex I-III activity was found in the ROT/Sal group, which was prevented by MB treatment (FIG. 5C). We interpret this as the result of ROS induced complex I/III damage, as previous publications suggested that complex III is the most sensitive component in the oxidative phosphorylation chain during stress. We also measured overall ROS in the brain extracts, and found that the ROT/Saline group had a significantly higher level of ROS compared to control groups. The increase of ROS by rotenone was also attenuated in the ROT/MB group to basal level (FIG. 5D).

Histological analysis was used for classical PD pathology. In the ROT/Sal group, inhibition of complex I resulted in degeneration of nigrostriatal dopaminergic neurons. With this dose and duration of rotenone exposure, animals demonstrated severe loss of dopaminergic neuron at the substantia nigra pars compacta, together with varying degrees of striatal dopaminergic denervation. Animals with dopaminergic neuron degeneration in substantia nigra were variable and correlate to their individual behavioral performance. Near complete loss of TH-positive cells in substantia nigra was observed in 5 out of 11 rotenone-treated animals with the remaining showed varying degrees loss of TH positive cells. Furthermore, TH staining of the ventral tegmental area (VTN) showed remarkable nerve fiber degeneration in ROT/Sal treated animals, a similar finding as observed in previously. On the other hand, in the ROT/MB animals, a sufficient amount of TH-positive cell and nerve fiber were observed in SNC and VTN, respectively.

To confirm that the loss of TH staining in dopaminergic neurons and nerve terminals was due to neurodegeneration, fluoro-jade B histochemistry was performed in brain sections from these animals. Fluoro-jade B-positive TH neurons were not found in either Veh/Sal group or the ROT/MB group. In contrast, fluoro-jade B-positive neurons were observed in the substantia nigra areas in 4 out of 11 ROT/Sal animals. We further examined the fluoro-jade B positive cells with double labeling of TH. Almost all fluoro-jade B positive cells were TH positive neurons. Together, double labeling of TH and fluoro-jade B conclusively demonstrated selective degeneration of nigra-striatal dopaminergic neurons in rotenone-infused animals, which was almost completely blocked by MB. In addition, ubiquitin-positive cytoplasmic inclusions were observed in rats with dopaminergic neuron degeneration. Such inclusions were frequently clustered in the cytoplasm of ubiquitin-positive cells, similar to those seen in Lewy bodies. In our study, 6 out 11 ROT/Sal rats showed ubiquitin positive aggregates, while such inclusion bodies were not found in any of the animals in the control group or the ROT/MB groups.

The neuroprotective action of MB was assessed in an ischemic stroke model of transient MCA occlusion in rats. Massive mitochondrial failure, indicated by reduced ETC complex I, III, and IV activities was observed at 24 hr after cerebral ischemia/reperfusion injury. By rerouting electron transfer which bypasses complex I and III blockage, a single dosage of MB at 500 μg/kg significantly reduced the ischemic lesion volume.

An embodiment of the invention is directed to the use of MB or similar compounds to provide neuroprotection using alternative electron transfer. Neuroprotection is a therapeutic approach that aims to prevent or attenuate neuronal degeneration and loss of function in neurological diseases. The alternative electron transfer through MB redox cycle bypasses the inhibition of ETC complex I and III, avoids over production of free radical, and provides neuroprotection in both chronic and acute neurological diseases. In an embodiment of the invention, MB or similar compounds function as an electron carrier and provide an alternative electron transfer along ETC, avoid ROS over production induced by ETC blockage, maintain mitochondrial function, and protect against neurodegeneration. In an animal model of Parkinsonism, MB was able to attenuate rotenone-induced motor deficits and nigral-dopaminergic neuronal degeneration. In an ischemic stroke model, MB significantly reduced cerebral ischemia reperfusion damage. In vivo studies indicate that MB attenuates complex I inhibition induced neurodegeneration in dopaminergic neurons, and provides protection against rotenone-induced Parkinson-like behavioral, neurochemical, and neuropathological features.

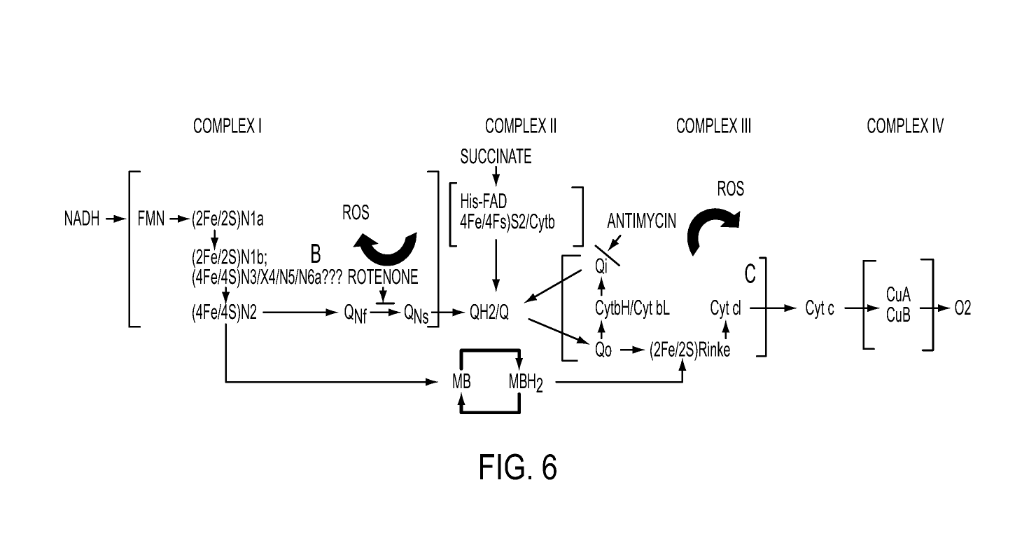

An embodiment of the invention is directed to the use of MB or similar compounds for the treatment of mitochondrial dysfunction related neurological diseases, such as PD and stroke using alternative electron transfer as a therapeutic approach. In determining the action of MB in ETC complex activities, MB functions as an alternative electron carrier. MB-mediated electron transfer is insensitive to either rotenone or antimycin A inhibition, suggesting that MB provides an alternative route for electron transfer. Upon the completion of the redox cycle (MB→MBH₂→MB), electrons from NADH are delivered to cyt c in an alternate route that is insensitive to complex I and III blockage. Such mechanism is further confirmed by a de novo synthesized MB derivative with the disabled redox center by N-acetylation. Acetyl-MB completely lost its ability to enhance electron transfer between complex I and III. The alternative electron transfer through MB prevents the “electron leakage” induced by complex I/III inhibition, and avoids the massive ROS production during complex I/III inhibition (FIG. 6).

An embodiment of the invention is directed to the use of MB or similar compounds for the treatment of Freiderich's Ataxia. The presence of MB greatly ameliorates the damaging effects of BSO in BSO induced FRDA fibroblast cell death, which mimics the disease condition, Friedrich's Ataxia, in vitro.

An additional embodiment of the invention is directed to the use of MB or similar compounds for the treatment of mitochondrial dysfunction related neurological diseases, such as Alzheimer's disease. Long term feeding of MB supplemented diet improves maximal performance of preclinical animal model of Alzheimer's disease, and improves cognitive functions of preclinical animal model of Alzheimer's disease.

The alternative electron transfer strategy blocks the over production of ROS generated by the inhibition of ETC complex I and III rather than neutralizing the free radical. In addition, MB increases oxygen consumption rate and decreases extracellular acidification rate, which prevents the superoxide production derived from the excessive oxygen supply during the reperfusion. In vivo studies have demonstrated that MB, as an alternative electron carrier, significantly decreases the cerebral ischemia reperfusion damage induced by transient focal cerebral ischemia.

In an embodiment of the invention, derivatives of methylene blue display varying levels of efficacy in protecting cells from death, ROS production and mitochondrial membrane potential collapse. As set forth in Table I below, a side chain at the N-motif reduces the EC50 around up to 1000-fold.

TABLE I
EC50 of methylene blue and its derivatives against cell death, ROS production, and
mitochondrial membrane potential collapse induced by glutamate in HT-22 cells

| | Protection | | Protection | | Protection against | |
|---|---|---|---|---|---|---|
| | against cell | | against | | Mitochondrial membrane | |
| | death | | ROS production | | potential collapse | |
| Methylene Blue Derivative | (EC50) | | (EC50) | | (EC50) | |
| 2-chlorophenothiazine | 4.57 | nM | 84.1 | nM | 45.1 | nM |
| phenothiazine | 19.0 | nM | 57.2 | nM | 21.2 | nM |
| toluidine blue | 1.75 | nM | 0.106 | nM | 1.54 | nM |
| methylene blue | 18.8 | nM | 20.4 | nM | 17.7 | nM |
| chlorpromazine | 2.19 | μM | 2.15 | μM | 3.27 | μM |
| promethazine | 2.06 | μM | 9.69 | μM | 214 | nM |
| neutral red | 5.84 | μM | 15.8 | μM | 8.96 | μM |
| Iminostilbene | 189 | nM | 484 | nM | 61.7 | nM |
| impramine | 51.9 | μM | 84.6 | μM | 29.1 | μM |

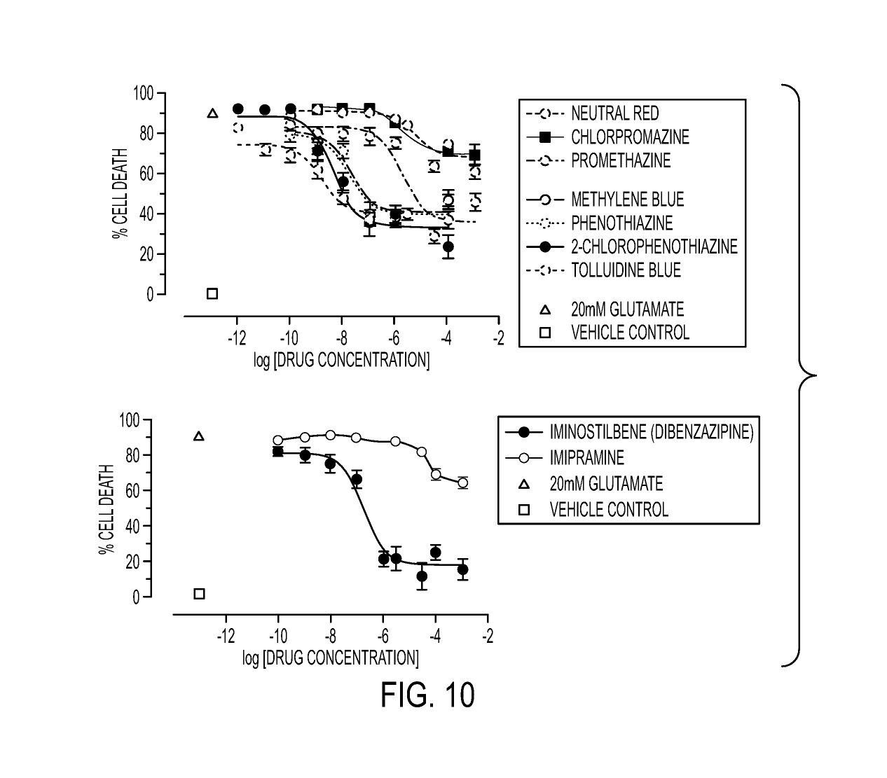

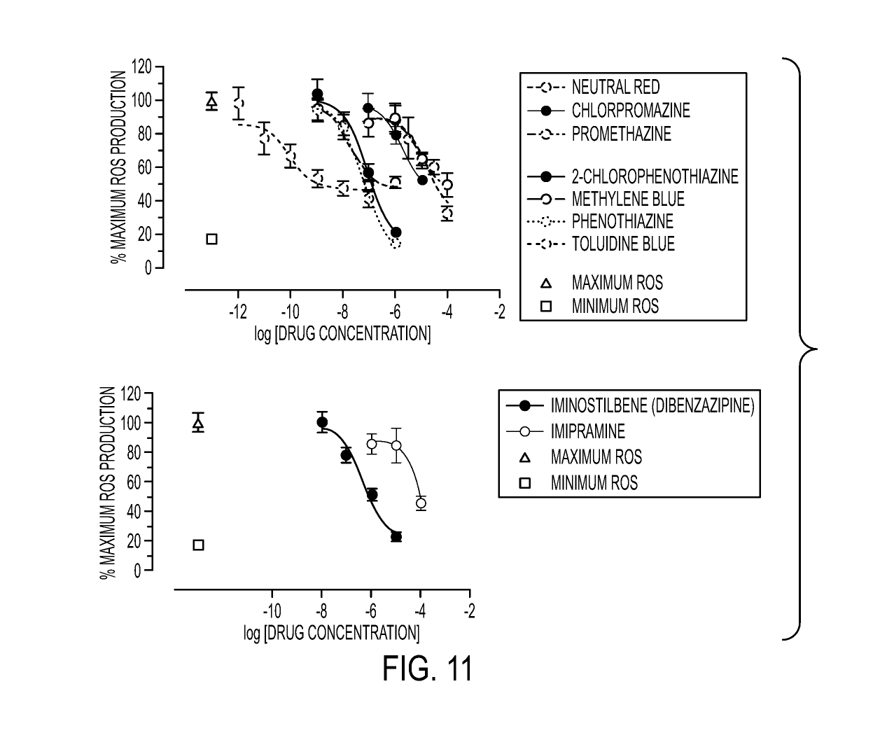

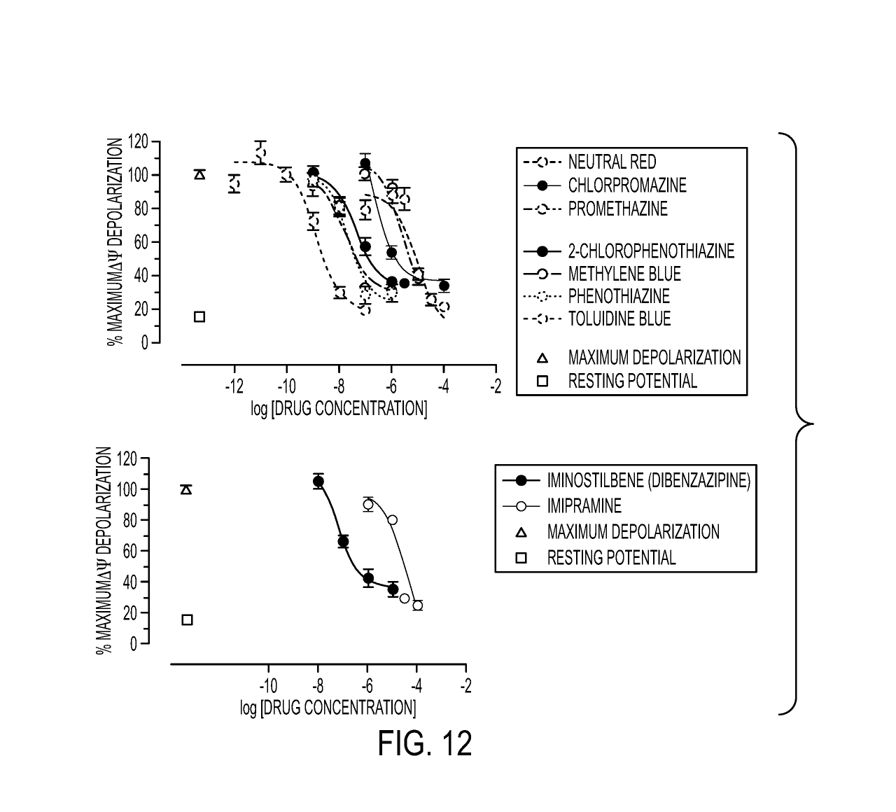

As shown in FIG. 10, a side chain at N-motif (Chlorpromazine, promethazine, imipramine) significantly decreases the potency and efficacy of the protective action. In addition, the S-motif is also critical as replacing S to N (Neutral Red) significantly decrease the potency and efficacy of the protection. Similarly, FIG. 11 shows the effect of methylene blue and its derivatives on reactive oxygen species production induced by glutamate in HT-22 cells, showing that a side chain at N-motif (Chlorpromazine, promethazine, imipramine) significantly decrease the potency and efficacy of the protective action. In addition, the S-motif is also critical as replacing S to N (Neutral Red) significantly decrease the potency and efficacy of the protection. FIG. 12 shows the effect of MB and its derivatives on mitochondrial membrane potential collapse induced by glutamate in HT-22 cells, showing that a side chain at N-motif (Chlorpromazine, promethazine, imipramine) significantly decrease the potency and efficacy of the protective action. In addition, the S-motif is also critical as replacing S to N (Neutral Red) significantly decrease the potency and efficacy of the protection.

As demonstrated in the Examples section, exemplary compositions of the present invention were shown to successfully treat and ameliorate a CNS-associated experimental disease condition of animal models, namely in a rotenone induced animal model of Parkinsonism and an animal model of ischemic stroke induced by middle cerebral artery occlusion, by attenuating the progress of the disease at various stages thereof as measured by qualitative observation of the pathological state of the animal models.

Each of the compositions described herein can therefore be utilized in any of the aspects of the present invention in a form of a pharmaceutically acceptable salt, a prodrug, a solvate and/or a hydrate thereof.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. The prodrug may also have improved solubility as compared with the parent drug in pharmaceutical compositions. Prodrugs are also often used to achieve a sustained release of the active compound in vivo. An example, without limitation, of a prodrug would be the methylene blue compound or analog, having one or more carboxylic acid moieties, which is administered as an ester (the “prodrug”). Such a prodrug is hydrolysed in vivo, to thereby provide the free compound (the parent drug). The selected ester may affect both the solubility characteristics and the hydrolysis rate of the prodrug.

The term “solvate” refers to a complex of variable stoichiometry (e.g., di-, tri-, tetra-, penta-, hexa-, and so on), which is formed by a solute and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

The beneficial characteristics of the compositions described herein render such compounds highly suitable for use in the treatment of the several medical conditions listed below, all of which involve mitochondrial dysfunction as a first or critical sign of loss-of-function:

Movement disorders: Parkinson's Disease (PD); Huntington's disease; Friedreich's ataxia (FRDA); Ataxia telangiectasia (AT); Amyotrophic lateral sclerosis (ALS); Fragile X-associated tremor/ataxia syndrome (FXTAS)

Cognitive dementia: Alzheimer's Disease; frontotemporal lobar degeneration; Vascular dementia; Down's syndrome; Mild Cognitive Impairment (MCI) and age-related cognitive decline.

Rapid injury: Brain traumatic injury; Brain stroke and functional recovery; Cyanide/mono-oxide toxication

Visual system disease: Glaucoma; Leber's hereditary optic neuropathy (LHON); Kearns Sayre syndrome; progressive external ophthalmoplegia (PEO)

Peripheral disease: Heart attack; Heart Failure, Diabetes, infection/septic shock

Energy utilization disorders: Diabetes, hot flash, body weight and appetite control; metabolic syndromes; for e.g., hot flashes are caused by an inability of the brain to take up and utilize glucose as an energy source. As a result, compounds that re-direct electrons within the mitochondria could be of benefit in providing the biological form of energy, ATP, in post-menopausal women.

Additionally, the compositions of the invention can thus be beneficially used to treat various oxidative stress associated diseases or disorders and/or related conditions including, without limitation, atherosclerosis, ischemia/reperfusion injuries, restenosis, hypertension, cancer, inflammatory diseases or disorders, acute respiratory distress syndrome (ARDS), asthma, inflammatory bowel disease (IBD), dermal and/or ocular inflammations, arthritis, metabolic diseases or disorders and diabetes.

The compositions of the invention can also be beneficially used to treat various CNS associated diseases, disorders or trauma, and/or related conditions including, without limitation, neurodegenerative diseases or disorders, strokes, brain injuries and/or trauma, multiple sclerosis, amyotrophic lateral sclerosis (ALS), Huntington's Disease, Parkinson's disease, Alzheimer's disease, autoimmune encephalomyelitis, AIDS associated dementia, epilepsy, schizophrenia, pain, anxiety, impairment of memory, decreases in cognitive and/or intellectual functions, deteriorations of mobility and gait, altered sleep patterns, decreased sensory inputs, imbalances in the autonomic nerve system, depression, dementia, confusion, catatonia and delirium.

As used herein, the phrase “therapeutically effective amount” describes an amount of the compound being administered which will relieve to some extent one or more of the symptoms of the disorder being treated, herein the medical conditions as detailed hereinabove. More specifically, a therapeutically effective amount means an amount of the composition of the invention which is sufficient and effective to prevent, alleviate or ameliorate some or all the symptoms of the medical condition or prolong the survival of the subject being treated.

The compositions described herein can be administered, for example, orally, rectally, intravenously, intraventricularly, topically, intranasally, intraperitoneally, intestinally, parenterally, intraocularly, intradermally, transdermally, subcutaneously, intramuscularly, transmucosally, by inhalation and/or by intrathecal catheter.

By being highly beneficial in treating certain medical conditions, the compositions described herein can be efficiently used for the preparation of a medicament for treating the abovementioned medical conditions.

In any of the aspects of the present invention, the compositions described herein, either alone or in combination with any other active agents, can be utilized either per se, or as a part of a pharmaceutical composition.

Hence, according to another aspect of the present invention, there are provided pharmaceutical compositions, which comprise, as an active ingredient, one or more of the compositions described above and a pharmaceutically acceptable carrier.

The pharmaceutical composition may further comprise an additional active ingredient being capable of treating the medical conditions, as detailed hereinabove.

As used herein a “pharmaceutical composition” or “medicament” refers to a preparation of one or more of the compositions described herein, with other chemical components such as pharmaceutically acceptable and suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, cyclodextrins, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the methylene blue compounds into preparations which, can be used pharmaceutically. Proper formulation is dependent upon the route of administration chosen.

For injection, the compositions of the invention may be formulated in aqueous solutions, preferably in physiologically compatible buffers such as Hank's solution, Ringer's solution, or physiological saline buffer with or without organic solvents such as propylene glycol, polyethylene glycol.

For transmucosal administration, penetrants are used in the formulation. Such penetrants are generally known in the art.

For oral administration, the compositions of the invention can be formulated readily by combining methylene blue or similar compounds with pharmaceutically acceptable carriers well known in the art. Such carriers enable the compositions of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the compositions may be dissolved or suspended in suitable liquids, such as fatty oils, liquid paraffin, or liquid polyethylene glycols. In addition, stabilizers may be added. All formulations for oral administration should be in dosages suitable for the chosen route of administration. Preferably, formulations for oral administration further include a protective coating, aimed at protecting or slowing enzymatic degradation of the preparation in the GI tract.

For buccal administration, the compositions may take the form of tablets or lozenges formulated in conventional manner.

For administration by inhalation, the compositions for use according to the present invention are conveniently delivered in the form of an aerosol spray presentation (which typically includes powdered, liquified and/or gaseous carriers) from a pressurized pack or a nebulizer, with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compositions of the invention and a suitable powder base such as, but not limited to, lactose or starch.

The compositions described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the methylene blue preparation in water-soluble form. Additionally, suspensions of the inventive compositions may be prepared as appropriate oily injection suspensions and emulsions (e.g., water-in-oil, oil-in-water or water-in-oil in oil emulsions). Suitable lipophilic solvents or vehicles include fatty oils such as sesame oil, or synthetic fatty acids esters such as ethyl oleate, triglycerides, liposomes or Cremophor® and various cremophor-like compounds (nonionic solubilizers and emulsifiers produced by reacting castor oil or other oils with ethylene oxide in various molar ratios). Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compositions to allow for the preparation of highly concentrated solutions.

Alternatively, the compositions may be in powder form for constitution with a suitable vehicle, e.g., sterile, pyrogen-free water, before use.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose, described hereinabove as a therapeutically effective amount.

Determination of a therapeutically effective amount is well within the capability of those skilled in the art, especially in light of the detailed disclosure provided herein.

For compositions used in the methods of the invention, the therapeutically effective amount or dose can be estimated initially from activity assays in animals. For example, a dose can be formulated in animal models, as demonstrated in the Examples section that follows, to achieve a circulating concentration range that includes the IC₅₀ as determined by activity assays.

The amount of a composition to be administered will, of course, be dependent on the subject being treated, the severity of the affliction, the manner of administration, the judgment of the prescribing physician, etc.

Thus, according to a preferred embodiment of the present invention, the pharmaceutical composition described herein is used in the treatment of a medical condition selected from the group consisting of a medical condition in which a CNS associated disease or, disorder or trauma, an oxidative stress associated disease or disorder, a disease or disorder in which neuroprotection is beneficial, and a medical condition at least partially treatable by the compositions of the invention.

Additional objects, advantages, and novel features of the present invention will become apparent to one ordinarily skilled in the art upon examination of the examples herein, which are not intended to be limiting. Additionally, each of the various embodiments and aspects of the present invention as delineated hereinabove and as claimed in the claims section below finds support in the specification.

1. A pharmaceutical composition, the composition comprising a therapeutically effective amount of a compound that can function as an alternative electron carrier.

2. The composition of claim 1, wherein said compound is methylene blue.

3. The composition of claim 1, wherein said compound is a methylene blue analog.

4. The composition of claim 1, wherein said methylene blue analog is leuco-methylene blue or acetyl-methylene blue.

5. The composition of claim 1, further comprising at least one pharmaceutically active agent in addition to methylene blue.

6. A method of treating a subject with at least one of a neurodegenerative condition, the method comprising administering to the subject a composition as defined in 1.

7. The method of claim 6 wherein the treatment is associated with the effect of the compound on the electron transport chain.

8. The method of claim 6, wherein the at least one of the neurodegenerative condition is selected from the group consisting of Parkinson's Disease (PD); Huntington's disease; Friedreich's ataxia (FRDA); Ataxia telangiectasia (AT); Amyotrophic lateral sclerosis (ALS); Fragile X-associated tremor/ataxia syndrome (FXTAS), Alzheimer's Disease; frontotemporal lobar degeneration; Vascular dementia; Down's syndrome; Brain traumatic injury; Brain stroke and functional recovery; Cyanide/mono-oxide toxication, Mild Cognitive Impairment (MCI) and age-related cognitive decline and combinations thereof.

9. A method of treating a subject with at least one of a visual system disease, the method comprising administering to the subject a composition as defined in 1.

10. The method of claim 9, wherein the at least one of the visual system disease is selected from the group consisting of Glaucoma; Leber's hereditary optic neuropathy (LHON); Kearns Sayre syndrome; progressive external ophthalmoplegia (PEO).

11. A method of treating a subject with at least one of a peripheral disease, the method comprising administering to the subject a composition as defined in 1.

12. The method of claim 9, wherein the at least one of the peripheral disease is selected from the group consisting of Heart attack; Heart Failure, Diabetes, infection/septic shock.

13. A method of treating a subject with at least one of a energy utilization disorder, the method comprising administering to the subject a composition as defined in 1.

14. The method of claim 9, wherein the at least one of the energy utilization disorder is selected from the group consisting of diabetes, hot flash, body weight and appetite control; and metabolic syndromes.
